(19) United States
(12) Patent Application Publication
(10) Pub. No.: US 2018/0142214 A1
(43) Pub. Date: May 24, 2018

(54) IN VITRO METHOD FOR SCREENING TESTING COMPOUND TO EVALUATE ITS POTENTIAL AS LIVER DRUG

(71) Applicant: Industrial Technology Research Institute, Hsinchu (TW)

(72) Inventors: Mei-Wei Lin, Hsinchu County (TW); Dian-Kun Li, Taichung City (TW); Ling-Mei Wang, Taipei City (TW); Ching-Huai Ko, Changhua County (TW); Chin-Pen Lai, Hsinchu County (TW); Chun-Min Liu, Hsinchu City (TW); Hsiang-Wen Tseng, New Taipei City (TW)

(73) Assignee: Industrial Technology Research Institute, Hsinchu (TW)

(21) Appl. No.: 15/861,625

(22) Filed: Jan. 3, 2018

Related U.S. Application Data
(62) Division of application No. 14/510,136, filed on Oct. 9, 2014.

(30) Foreign Application Priority Data
Dec. 11, 2013 (TW) 102145617

(51) Int. Cl. C12N 5/09; G01N 33/50; C12N 5/071

(52) U.S. Cl. CPC C12N 5/0693; G01N 33/5067; C12N 5/067; G01N 33/5011

(57) ABSTRACT

An in vitro method for screening a testing compound to evaluate its potential as a liver drug of the disclosure is provided. The method includes applying the testing compound to cells of an isolated human liver tumor cell line, named as ITRI-H28, measuring a cell viability of the cells and determining the effect of the testing compound on the cells by calculating a half maximal inhibitory concentration (IC₅₀) of the testing compound.

### CROSS-REFERENCE TO RELATED APPLICATION

This application is a divisional application of and claims the priority benefit of U.S. application Ser. No. 14/510,136, filed on Oct. 9, 2014, now pending, which claims the priority benefit of Taiwan application serial no. 102145617, filed on Dec. 11, 2013. The entirety of each of the above-mentioned patent applications is hereby incorporated by reference herein and made a part of this specification.

### BACKGROUND

### Technical Field

The technical field relates to an isolated human liver tumor cell line and a method of an agent screening.

### Description of Related Art

Compared with treatments of other cancers, a response rate of liver cancer to a traditional chemotherapy, such as doxorubicin and cisplatin, is less than 20%. Therefore, the treatment of liver cancer is very limited and is yet to be confirmed in clinical trials to extend a patient survival time by traditional chemical drugs. Further, a targeted drug of liver cancer, Sorafenib (Nexavar), approved by the U.S. Food and Drug Administration in 2007 only extends the patient survival time of three-month in phase III of human clinical trials. Hence, a drug having efficacy in the liver cancer treatment urgently needs to be developed.

Pathogenesis of liver cancer is quite complex, the main causes are inflections of hepatitis B virus (HBV) and hepatitis C virus (HCV), aflatoxin, alcohol, and other possible causes of cirrhosis. Hepatitis C is considered as the most main cause of liver cancer in the countries of United States, Japan, etc. Therefore, with the increased incidence of liver cancer worldwide each year, studies of the pathogenesis of HCV-related hepatocellular carcinoma (HCV-related HCC) and developments of HCV-related HCC drugs are urgent issues. However, with an overview of liver tumor cell lines preserved in the internationally renowned cell repository centers, the American Type Culture Collection (ATCC) and Japanese Collection of Research Bioresources (JCRB), most of the liver tumor cell lines are HBV-related or HBV-negative. A successful establishment of a liver tumor cell line derived from a liver tumor tissue of a HCV-related HCC patient is not yet achieved. Therefore, HCV-related liver tumor cell lines are urgently needed in this field for research so as to facilitate the developments of the HCV-related HCC drugs by testing and screening, which can make up a deficiency of a development platform of the liver cancer drugs.

### SUMMARY

The disclosure relates to an in vitro method for screening a testing compound to evaluate its potential as a liver drug, in which an isolated human liver tumor cell line is used to perform the screening, thereby developing the liver drugs related to liver cancer, liver failure, or liver cancer stem cell.

An in vitro method for screening a testing compound to evaluate its potential as a liver drug of the disclosure includes the following steps. First, the testing compound is applied to cells of an isolated human liver tumor cell line, named as ITRI-H28. A cell viability of the cells is measured. Next, the effect of the testing compound on the cells is determined by calculating a half maximal inhibitory concentration (IC₅₀) of the testing compound.

To make the aforementioned more comprehensible, several embodiments accompanied with drawings are described in detail as follows.

### BRIEF DESCRIPTION OF THE DRAWINGS

The accompanying drawings are included to provide a further understanding of the disclosure, and are incorporated in and constitute a part of this specification. The drawings illustrate exemplary embodiments of the disclosure and, together with the description, serve to explain the principles of the disclosure.

FIG. 1 reveals a morphology of a monolayer-cell of a human liver tumor cell line ITRI-H28, which is observed under a phase contrast microscope with a magnification of 40× according to one embodiment of the disclosure.

FIG. 2 shows a growth curve of a human liver tumor cell line ITRI-H28 according to one embodiment of the disclosure.

FIG. 3 shows a cell viability of the human liver tumor cell line ITRI-H28 in the groups after performing treatments of Sorafenib in different concentrations according to one embodiment of the disclosure.

FIG. 4 shows a luciferase expression of a human liver tumor cell line ITRI-H28 transfected by lentiviral gene vectors according to one embodiment of the disclosure.

FIG. 5 reveals a morphology of a human liver tumor cell line ITRI-H28 cultured in ultra low attachment flask after 5 days, which is observed under a phase contrast microscope according to one embodiment of the disclosure.

FIG. 6 shows a relationship between tumor incidence and injection time, after implanting 100 ITRI-H28 cells and 1000 ITRI-H28 cells in sever combined immune deficient mice according to one embodiment of the disclosure.

### DESCRIPTION OF THE EMBODIMENTS

Below, exemplary embodiments will be described in detail with reference to accompanying drawings so as to be easily realized by a person having ordinary knowledge in the art. The inventive concept may be embodied in various forms without being limited to the exemplary embodiments set forth herein. Descriptions of well-known parts are omitted for clarity, and like reference numerals refer to like elements throughout.

In the disclosure, a human liver tumor cell line is cultured after isolating from a liver tumor tissue, and which is named as ITRI-H28. The ITRI-H28 cell line was deposited in the Food Industry Research and Development Institute with an accession number BCRC960457. According to a cell appearance morphology, a growth curve, an isoenzymes analysis, a genotyping, a cytogenic analysis, secreted proteins, and a secreted enzyme activity of the ITRI-H28 cell line, it shows that the human liver tumor cell line is, indeed, derived from a liver tissue of a HCV-related HCC patient. The human liver tumor cell line has tumor cell properties and is a newly established cell line. A result of a cancer stem cell property analysis reveals that the ITRI-H28 cell line highly expresses cancer stem cell molecules, is capable of forming a spheroid structure, and is tumorigenic to the sever combined immune deficient mice, which indicates that the ITRI-H28 has a cancer stem cell potential. Furthermore, a result of an agent screening reveals that the established human liver tumor cell line ITRI-H28 has a susceptibility to cancer drugs, which is applicable in a cancer drug screening. In addition, an established luciferase expression system is employed for performing an in vivo image observation through an in vivo imaging system (IVIS) during a construction of a mice xenograft model. For drug developments and efficacy evaluations of liver cancer, the disclosure simultaneously provides an in vivo analysis platform and an in vitro analysis platform that can be used in researches of a mechanism of hepatitis C viral carcinogenesis.

### Experiment 1

Establishment of a Human Liver Tumor Cell Line ITRI-H28

In the disclosure, the human liver tumor cell line ITRI-H28 is cultured after isolating from a liver tumor tissue of a HCV-related HCC patient. The ITRI-H28 cell line was deposited in the Food Industry Research and Development Institute with an accession number BCRC960457 on Dec. 14, 2012.

After obtaining the liver tumor tissue from the HCV-related HCC patient, the tissue is immersed in Hank's balanced salt solution (HBSS). Next, the liver tumor tissue is cut into tumor tissue segments with a size of 5 mm*5 mm or smaller by a sterile scalpel. Then, under a cultured environment of 37° C., the liver tumor tissue segments are treated with a three-enzyme combined solution including dispase, collagenase, and DNase, so as to digest connective tissues, thereby releasing the liver cancer cells from the tissue at a lower damage level. Subsequently, a digestive solution containing the liver cancer cells is filtered by a filter of a 100 um mesh. A cell suspension collected after filtering is transferred to a 50 ml centrifuged tube and is centrifuged at 1000 rpm for 5 minutes. A supernatant is removed. Next, 5 ml of red blood cell (RBC) lysis buffer is added and is reacted with the cell suspension for 3 minutes to remove red blood cells, and then the cell suspension is further centrifuged at 1000 rpm for 5 minutes to remove a supernatant. The liver cancer cells are then suspended again in a 10 ml culture medium. The cells are placed in an MEM-alpha (Gibco, 12561-056) culture medium containing 10% serum and are cultured in a constant-temperature cell culture chamber of 5% CO₂ at 37° C. for primary culture.

Subculture

When a bottom of a cell culture plate is covered with the cells of the primary culture, an old culture medium is removed, and the plate is washed with PBS buffer. Then, trypsin is added to the cell culture plate for digestion, which decomposes attachment proteins between cell-cell or on sidewalls of the cell culture plate. Thus, the cells are detached from the sidewalls of the cell culture plate. By a ratio of 1:3-1:4 for the cells to the culture medium, a new culture medium is added for performing a subsequent culture. After that, the subculture is performed every four days so as to obtain the ITRI-H28 cell line having uniform morphology and higher purity.

Therefore, the human liver tumor cell line is successfully established, which the accession number thereof in the Food Industry Research and Development Institute is BCRC960457, and subsequent tests are performed. On the other hand, the human liver tumor cell line of the disclosure is not limited to the ITRI-H28 cell line described herein. A cell line is obtained by subcloning or monocloning derived from the ITRI-H28 cell line, or a cell line has any identifiable characteristics similar to the ITRI-H28 cell line, they fall within the scope of claims in the disclosure.

Particularly, in a process of a purification of the cancer cells, it often causes a large number of cells to be damaged. In addition, since the cancer cells are isolated from the tumor tissues, a growth of the cancer cells is significantly influenced by the mixed and mingled cells (i.e., lymphocytes, fibroblasts, tumor necrosis cells). It makes the isolation and the purification of the cancer cells quite difficult. Therefore, in the past, a success rate of the cancer cells in primary culture is low. In order to prevent that the other mixed and mingled non-cancer cells influence the cancer cell growth and further impact an establishment of the tumor cell line, we employ a method of extended subculture time. Since tumor cells do not have the characteristic of “contact inhibition growth”, and a growth of normal cells (such as fibroblasts) is regulated by the contact inhibition growth, a growth of mixed and mingled fibroblasts is gradually stopped due to the contact inhibition. As a result, the growth of the cancer cells gradually becomes more significant. Therefore, not only the doubts for having the mixed and mingled fibroblasts in the process of establishing the cancer cells are significantly reduced, but also the cancer cell growth is advantaged, therefore, the human tumor liver cell line ITRI-H28 is successfully established.

### Experiment 2

Observation of a Cell Appearance Morphology of a Human Liver Tumor Cell Line ITRI-H28

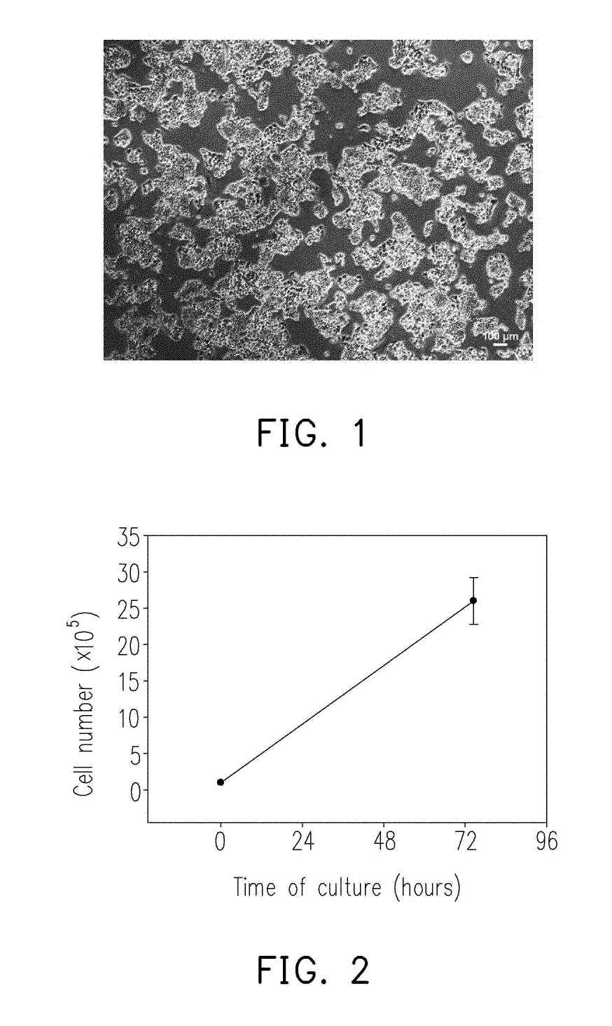

A human liver tumor cell line ITRI-H28 is placed under a phase contrast microscope (Nikon Eclipse Ti-S) to observe the morphology with a magnification of 40×, and the result is shown in FIG. 1.

Please refer to FIG. 1, which reveals a morphology of a monolayer-cell of the human liver tumor cell line ITRI-H28, which is observed under the phase contrast microscope with a magnification of 40×. According to FIG. 1, the monolayer-cell of the human liver tumor cell line ITRI-H28 attaches to a culture plate coated with 1% collagen and shows the cell characteristics of large karyoplasm ratio, poor cell extensibility, and strong refraction. The above characteristics indicate that the ITRI-H28 is a poorly differentiated liver cancer cell line.

### Experiment 3

Determination of a Growth Curve of a Human Liver Tumor Cell Line ITRI-H28

A test is used to determine a regular growth curve of the human liver tumor cell line ITRI-H28, which is a continuously self-subculture of twenty-sixth generation. 1×10⁵ of the ITRI-H28 cell lines are seeded in a 6-well plate, an MEM-alpha culture medium containing 10% serum is added to the plate, and a culture is performed in a constant-temperature cell culture chamber with 5% CO₂ at 37° C. Every 72-hour, the plate is taken from the chamber and is placed under a microscope for counting a cell number. A population doubling time of the cells is calculated and is shown in FIG. 2.

FIG. 2 shows a growth curve of a human liver tumor cell line ITRI-H28. Please refer to FIG. 2, the population doubling time of the human liver tumor cell line ITRI-H28 is 15.96 hours. Namely, a growth speed rate of the ITRI-H28 cell line is rapid, which is one of the characteristics of the cancer cells.

### Experiment 4

Isoenzyme Analysis of a Human Liver Tumor Cell Line ITRI-H28

In order to confirm the ITRI-H28 cell line is derived from humans without contaminating by other cells, the experiment employs AuthentiKit™ (Innovative Chemistry Marshfield, Mass.) and a manufacturer instruction thereof to perform isoenzyme analysis on seven isoenzymes of the ITRI-H28 cell line. Accordingly, corrected migration distances (CMD) of electrophoretic band of the isoenzymes are calculated. The seven isoenzymes are nucleoside phosphorylase (NP), glucose-6-phosphate dehydrogenase (G6PD), malate dehydrogenase (MD), mannose phosphate isomerase (MPI), peptidase B (PepB), aspartate aminotransferase (AST) and lactate dehydrogenase (LD). Meanwhile, a human 293 HEK cell line is treated as a human control group, and a porcine PK 15 cells line is treated as a porcine control group. General data of the electrophoretic band CMDs of the human isoenzymes serving as standard references is also provided, and results are shown in Table 1.

TABLE 1

| | Corrected Migration distance (CMD) | | | | | | |
|---|---|---|---|---|---|---|---|
| Cell | NP | G6PD | MD | MPI | PepB | AST | LD |
| Porcine control | 11.5 | 10.8 | 13.2 | 15 | 25.8 | 13 | 1.0 |
| group (PK15) | | | | | | | |
| Human control | 12.8 | 12.2 | 8.6 | 12 | 12.9 | 15.1 | −5.0 |
| group (293 HEK) | | | | | | | |
| Human standard | 12.9 | 14.5 | 8.3 | 12.9 | 12.4 | 15 | −5.6 |
| reference | | | | | | | |
| ITRI-H28 | 13.4 ± 2 | 15.1 ± 2 | 8.5 ± 2 | 12.5 ± 2 | 12.0 ± 2 | 15.1 ± 2 | −5.0 ± 2 |

The electrophoretic band CMDs of the isoenzymes of the ITRI-H28 cell line, the 293 HEK cell line, and the PK 15 cell line are shown in Table 1. According to Table 1, by comparing zymography of the seven isoenzymes of the ITRI-H28 cell line, the 293 HEK cell line, and the PK 15 cell line, the ITRI-H28 cell line and the human 293 HEK cell line have similar zymography. In addition, the zymography of the ITRI-H28 cell line is significantly different from that of the PK 15 cell line. It can be seen that the ITRI-H28 cell line and the human 293 HEK cell line share the same origin. Namely, the ITRI-H28 cell line is classified in human cells.

### Experiment 5

Genotyping of a Human Liver Tumor Cell Line ITRI-H28

In order to confirm the human liver tumor cell line ITRI-H28 is derived from a liver tumor tissue resected from a liver cancer patient, the experiment employs AmpFISTR Identifier PCR Amplification Kit to analyze the ITRI-H28 cell line DNA so as to perform a DNA fingerprinting confirmation of the ITRI-H28 cell line. In detail, fifteen short tandem repeats (SRT) of the ninth (p9) and forty-sixth (p46) generations of the ITRI-H28 cell line and segments of allelic pattern of a X-Y homologous gene (amelogenin) are analyzed. The fifteen SRTs of the ITRI-H28 cell line are D831179, D21S11, D7S820, CSF1PO, D3S1358, TH01, D13S317, D16S539, D2S1338, D19S433, vWA, TPOX, D18S51, D5S18, and FGA. Meanwhile, the same method is adopted to analyze allelic patterns of the resected liver tumor tissue in Experiment 1, and results are shown in Table 2.

TABLE 2

| | | Resected | ITRI-H28 | ITRI-H28 |
|---|---|---|---|---|
| | | liver tumor | cell line | cell line |
| | Chromosomal | tissue, allelle | (p9), allelle | (p46), allelle |
| STR-locus | Location | (1, 2) | (1, 2) | (1. 2) |
| D8S1179 | 8 | 14, 15 | 14, 15 | 14, 15 |
| D21S11 | 21q11.2-q21 | 29, 33.2 | 29, 33.2 | 29, 33.2 |
| D7S820 | 7q11.21-22 | 10, 11 | 10 | 10 |
| CSF1PO | 5q33.3-34 | 10, 13 | 10, 13 | 10 |
| D3S1358 | 3q | 15, 16 | 15, 16 | 15, 16 |
| TH01 | 11p15.5 | 9 | 9 | 9 |
| D13S317 | 13q22-31 | 8, 10 | 10 | 10 |
| D16S539 | 16q24-qter | 9, 11 | 9 | 9 |
| D2S1338 | 2q35-37.1 | 19 | 19 | 19 |
| D19S433 | 19q12-13.1 | 13 | 13 | 13 |
| vWA | 12q12-pter | 14, 16 | 14, 16 | 14, 16 |
| TPOX | 2q23-2per | 8, 11 | 8, 11 | 8, 11 |
| D18S51 | 18q21.3 | 17 | 17 | 17 |
| D5S818 | 5q21-31 | 10, 11 | 10, 11 | 10, 11 |
| FGA | 4q28 | 21, 26 | 26 | 26 |
| Amelogenin | X: p22.1-22.3 | X, Y | X, Y | X, Y |
| | and Y: p11.2 | | | |

The Genotyping data of the ITRI-H28 cell line and the resected liver tumor tissue are shown in Table 2. According to Table 2, the genotyping of the ITRI-H28 cell line and the liver tumor tissue are identical, unique and consistent, where the STR-PCR pattern does not exist in the existing data library. Therefore, the ITRI-H28 cell line is, indeed, a novel cell line derived from the liver tumor tissue resected from the patient of liver cancer.

### Experiment 6

Cytogenic Analysis of a Human Liver Tumor Cell Line ITRI-H28

The cytogenic analysis of the ITRI-H28 cell line is performed by the Center of Medical Genetics, Changhua Christian Hospital. In 20 cells examined, a variation of chromosome numbers is in a range of 77-84 (tetraploid), which reveals an abnormal chromosome number of the cells. Meanwhile, the following abnormalities related to the chromosome numbers and structures are found in the observation. These abnormalities include (1) rearrangement of chromosomes X and 17; (2) loss of chromosomes Y, 1, 2, 3, 5, 8, 9, 11, 12, 13, 14, 15, 16, 18, 19, 20, 21, and 22; (3) extra copies of chromosomes 5, 6, 17, and 22; (4) an isochromosome as a long arm of chromosome 8; (5) derivative chromosome 19 formed by translocation (t(14,19)) of chromosomes 14 and 19; and (6) a presence of 7-14 marker chromosomes.

On the other hand, Spectral karyotyping probe kit (ASI, Inc.) is employed to perform a spectral karyotyping (SKY) analysis on the ITRI-H28 cell line. In 10 metaphase cells examined, chromosomal pattern of each of the cells is unique. Furthermore, consistent variations of karyotyping is reported as following: 80-87, XXYY, der(1)t(1;16)×2, +1, −2, −3, der(4)t(1;4;8), −4, −6, der(7)t(7;14)×2, +7, +7, +7, −9, +11, −12, −13, −13, −14, −14, −15, −16, der(17)t(17;19), der(18)t(9;18), −18, der(19)t(14;19), −19, −19, +20, and −21[cp 10].

According to the above experimental results, abnormal separations of the chromosomes and multiple sets of the chromosomes reveal that the human liver tumor cell line ITRI-H28 has the cancer cell properties.

### Experiment 7

Function of Secreting Proteins of a Human Liver Tumor Cell Line ITRI-H28

Retrospect to the clinical records of a HCV-related HCC patient which the ITRI-H28 cell line is derived from, high expression level of alpha-fetoprotein (APP) and anti-HCV antibodies in a serum of the patient is found. It indicates that the liver cancer patient had been infected with HCV. However, extracting RNA of a supernatant and a cell lysate of the ITRI-H28 cell line is used to perform a virus detection, no HCV RNA is detected. Namely, the current ITRI-H28 cell line carrying no hepatitis C virus is confirmed.

A secretion analysis experiment of AFP and albumin of the human liver tumor cell line ITRI-H28 is performed. In the experiment, 2×10⁵ ITRI-H28 cells are seeded in a 6-well plate, an MEM-alpha culture medium containing 10% serum is added to the plate, and a culture is performed in a constant-temperature cell culture chamber with 5% CO2 at 37° C. Next, every 24-hour and 48-hour, the plate is taken from the chamber and is checked with the amount of AFP and albumin in the supernatant of the ITRI-H28 cell line. Meanwhile, a Huh-7 cell line having the same cell number is used as a control group. Results are shown in Table 3.

TABLE 3

| | Albumin (ng/ml) | | AFP(ng/ml) | |
|---|---|---|---|---|
| Cell | 24 hours | 48 hours | 24 hours | 48 hours |
| Huh-7 | 74.4 ± 1.7 | 157.0 ± 8.0 | 58.4 ± 0.1 | 63.3 ± 0.8 |
| ITRI-H16 | 153.7 ± 15.2 | 254.0 ± 26.8 | 1.4 ± 0.1 | 4.0 ± 0.1 |

The secreted amount of albumin and AFP of the ITRI-H28 cell line and the Huh-7 cell line are shown in Table 3. According to Table 3, the ITRI-H28 cell line still expresses AFP and albumin after being cultured in vitro for a time period, wherein albumin is synthesized by the liver cells. It is therefore known that the ITRI-H28 cell line has functions of synthesis and release of albumin. According to the above results, the ITRI-H28 cell line still has the function of expressing AFP and albumin after being cultured in vitro for a time period.

### Experiment 8

Application of a Human Liver Tumor Cell Line ITRI-H28 on an Agent Screening

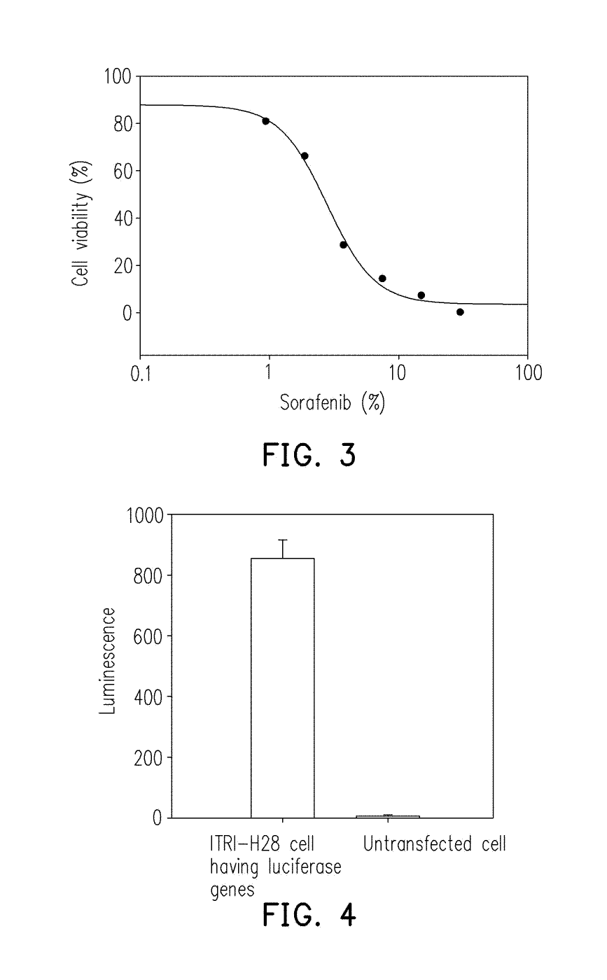

In the experiment, a plurality of plates are prepared with 1×10⁴ ITRI-H28 cells seeded respectively thereon and divided into a plurality of groups, and the groups are treated with different concentrations of Sorafenib, such as 30, 15, 7.5, 3.75, 1.88, and 0.94 uM. Next, after a 48-hour treating period, a cell viability of each of the groups is measured by an Alarmablue measurement assay, and results are shown in FIG. 3.

FIG. 3 shows the cell viability of the human liver tumor cell line ITRI-H28 in each of the groups after treating with Sorafenib in different concentrations. A calculation is done according to the results of FIG. 3, a half maximal inhibitory concentration (IC₅₀) of Sorafenib in the ITRI-H28 cell line are about 2.79±0.41, and a statistical P value is 0.02. According to the above results, the human liver tumor cell line ITRI-H28 has a susceptibility to the HCV-related HCC drug, which is able to use in a HCV-related HCC drug screening. Furthermore, according to the above experiments, the human liver tumor cell line ITRI-H28 has cancer cell properties, which can be used widely in a liver drug screening.

### Experiment 9

Establishment of a Vector Expression System of a Human Liver Tumor Cell Line ITRI-H28

In the experiment, a 6-well plate seeded with 2×10⁵ of the seventh generation of the ITRI-H28 cell lines is prepared. Next, a transfection solution containing 5 ug/ml of polybrene is used as a culture medium. The transfection solution is added into the ITRI-H28 cells with a ratio of every 1000-cell to 1 ul-transfection solution (Firefly Luciferase (FLuc) Lentivirus with Puro Selection). The plate is placed in a constant-temperature cell culture chamber with 5% CO₂ at 37° C. for culture. After a 16-hour transfection, the transfection solution is replaced by the original cell culture medium, and puromycin with a concentration of 5 ug/ml is used for performing a screening. After subculturing two generations in the culture medium containing puromycin, a ONE-Glo™ Luciferase assay is performed to examine luminescence of the cells, wherein untransfected third and fifth generations of ITRI-H28 cells are mixed and used as control groups. The results are shown in FIG. 4. The luminescence of non-transfection cells is negligibly small.

Please refer to FIG. 4, which shows luminescence measured in the human liver tumor cell line ITRI-H28 transfected by lentiviral gene-containing vectors. According to FIG. 4, an average luminescence of the transfected ITRI-H28 cell line and the untransfected ITRI-H28 cell line is 855.1±61.1 and 4.0±2.8, respectively. Namely, the ITRI-H28 cell line is capable of expressing luciferase genes.

### Experiment 10

Expressing Cancer Stem Cell Molecules of a Human Liver Tumor Cell Line ITRI-H28

In the experiment, using markers specific for cancer stem cells as targets, the markers are CD13, CD24, CD44, CD133, EpCAM, and OV6. The markers on surfaces of the ITRI-H28 cells are dyed via an immunofluorescence staining. Next, a number of the dyed ITRI-H28 cells are detected by a flow cytometer so as to obtain a cell ratio (%) of the cells expressing specific markers to the total cells, and results are shown in Table 5 as below. The expression of vimentin is treated as a control group.

TABLE 4

| | Ratio of cells expressing specific |
|---|---|
| Marker | markers to total cells (%) |
| CD13 | 93.89 |
| CD24 | 40.38 |
| CD44 | 92.82 |
| CD90 | 2.09 |
| CD133 | 1.93 |
| EpCAM | 98.84 |
| CD19 | 20.35 |
| Vimentin | 21.46 |

A level of the cancer stem cell markers expressed on the surfaces of the ITRI-H28 cell line is shown in Table 4. In Table 4, CD 13, CD44, and EpCAM are expressed by 90% of the ITRI-H28 cells or more. According to the above results, the ITRI-H28 cell line has a cancer stem cell potential.

### Experiment 11

Morphology of Cancer Stem Cells of a Human Liver Tumor Cell Line ITRI-H28

The cancer stem cells are a group of self-renewal cells existing in a tumor. Currently, the cancer stem cells not only play an important role in proliferation of the tumor, but also play an important role in treatment resistance and cancer recurrence. Generally speaking, a sphere formation of stem cells is a phenotype of the cancer stem cells and is evaluated.

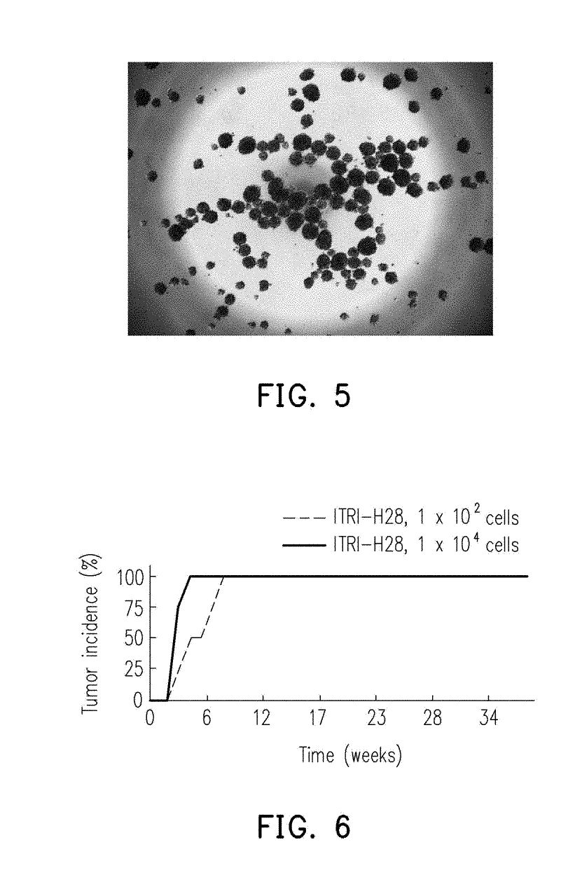

In the experiment, 2×10⁵ ITRI-H28 cell lines are seeded in an ultra low attachment flask, and after the MEM-alpha culture medium is added to the flask, a suspended culture is performed in a constant-temperature cell culture chamber with 5% CO₂ at 37° C. for 5 days. After a 5-day culturing period, the ITRI-H28 cell lines are placed under a phase contrast microscope (Nikon eclipse Ti-S) to observe cell morphology with magnification of 100×. The results are shown in FIG. 5.

FIG. 5 reveals the morphology of the human liver tumor cell line ITRI-H28 cultured in the ultra low attachment flask for 2 days, which is observed under the phase contrast microscope. According to FIG. 5, the ITRI-H28 cell line forms the spheroid-like structure of stem cell in the ultra low attachment flask. Therefore, the ITRI-H28 cell line has a cancer stem cell potential.

### Experiment 12

Carcinogenicity of a Human Tumor Cell Line ITRI-H28 in Sever Combined Immune Deficiency Mice

First, sever combined immunodeficiency mice (SCID mice) are prepared, which are 6-8 weeks of female mice purchased from BioLASCO Taiwan (stock: CB17/Icr-Prkdcscid/CrlBltw). Next, 100 ITRI-H28 cells and 1000 ITRI-H28 cells are respectively injected subcutaneously into the SCID mice, in which the ITRI-H28 cells are the mixing cells of the seventh generation and the fifteenth generation. After injecting the ITRI-H28 cells to the SCID mice, a volume of subcutaneous tumor of the SCID mice is measured three times a week so as to estimate a tumor incidence in the SCID mice.

Please refer to FIG. 6, which shows a graph of a relationship between tumor incidence and injection time, after injecting 100 ITRI-H28 cells and 1000 ITRI-H28 cells into the SCID mice. Generally speaking, tumorigenicity is one of important evaluation criteria of the cancer stem cells. Namely, as a small amount of cancer cells is capable of forming a new tumor, it indicates that the cancer stem cells have the ability of self-renewal and proliferation. According to FIG. 6, when 10000 ITRI-H28 cells are injected into the SCID mice, the tumor incidence after 6 weeks is up to 100%. When 100 ITRI-H28 cells are injected into the SCID mice, the tumor incidence after 9 weeks is up to 100%. It can be seen that the ITRI-H28 cells has high tumorgenicity which belongs to the cancer stem cell potential.

Through the above experimental results, the ITRI-H28 cell line is successfully isolated under a particular isolation condition. Moreover, with a method of extended subculture time, the ITRI-H28 cell line is capable of continuously and stably culturing and subculturing in vitro. The characteristics and functions of the liver cancer cell are retained, for instances, productions of alpha-fetoprotein and expressions of albumin of the liver cells. In addition, the ITRI-H28 cell line has the morphology of the cancer stem cells. A result of immunity analysis indicates that the ITRI-H28 cell line expresses high levels of the cancer stem cell markers, CD13, CD44, and EpCAM. Particularly, by analyzing the carcinogenicity of the ITRI-H28 cell line to SCID mice; it is found that the small amount of the ITRI-H28 cells has the ability to induce tumor growth in the mice. In other words, the ITRI-H28 cell line has a higher degree of carcinogenicity than other liver caner cell lines (such as Huh-7, PCLUPRF/5, HepG2, and Hep3B). It shows the ITRI-H28 cell line is a cell line having the cancer stem cell potential. On the other hand, for the drug developments, the ITRI-H28 cell line provides an in vitro analysis platform for evaluating the therapeutic efficacy HCV-related HCC drug. According to the above characteristics of the ITRI-H28 cell line, the ITRI-H28 cell line is also suitable for applications on a drug screening of drugs for liver failure and liver cancer stem cell.

The ITRI-H28 cell line is an established liver tumor cell line derived from the tissue of the HCV-related HCC patient, which can make up the deficiency of a drug development platform of only HBV-related or HBV-negative liver cancer. The ITRI-H28 is expected to play an important role in the field of HCV-related HCC research. By understanding the physiological characteristics of the IRTI-H28 cell line and the molecular-medicine characteristics thereof through the series of analyses as above, it is know that the ITRI-H28 cell line is capable of being used widely in the researches of mechanism of hepatitis C viral carcinogenesis, screening drugs for liver failure and liver cancer stem cell, liver cancer drug development, drug development for liver cancer stem cell, and drug metabolism.

In summary, the human liver tumor cell line of the disclosure is a first established cell research material derived from the tissue of the HCV-related HCC patient, and which is used as the research platform of the mechanism of HCV-related HCC and the drug screening related to liver cancer. In detail, the physiological properties and the molecular medicine characteristics of the IRTI-H28 cell line are studied in the series of analyses in above, which includes that the ITRI-H28 cell line is capable of being cultured in an extended subculture time thereby continuously and stably proliferating and subculturing, expressing vectors, and having the cancer stem cell potential. Therefore, the ITRI-H28 cell line is expected to be used widely in the field of HCV-related HCC research so as to improve the chance of cure for HCV-related HCC.

Base on the above, the isolated human liver tumor cell line of the disclosure is the cell line isolated from the tissue of the HCV-related HCC patient, and which has the liver cancer cell properties and the cancer stem cell potential. Therefore, the isolated human liver tumor cell line of the disclosure is the suitable research material of the mechanism of HCV-related HCC, and which is used as a drug screening platform of liver cancer or liver failure.

It will be apparent to those skilled in the art that various modifications and variations can be made to the disclosed embodiments without departing from the scope or spirit of the disclosure. In view of the foregoing, it is intended that the disclosure covers modifications and variations provided that they fall within the scope of the following claims and their equivalents.

### Biological Material Deposit

An isolated human liver tumor cell line of the disclosure is named as ITRI-H28. The ITRI-H28 cell line was deposited in the Food Industry Research and Development Institute with an accession number BCRC960457 on Dec. 14, 2012.

What is claimed is:

1. An in vitro method for screening a testing compound to evaluate its potential as a liver drug, comprising:
(a) applying the testing compound to cells of an isolated human liver tumor cell line, named as ITRI-H28;
(b) measuring a cell viability of the cells; and
(c) determining the effect of the testing compound on the cells by calculating a half maximal inhibitory concentration (IC₅₀) of the testing compound.

2. The in vitro method as recited in claim 1 further comprising comparing the effect of the testing compound on the cells to a control.

3. The in vitro method as recited in claim 1, wherein Sorafenib is used as the control.

4. The in vitro method as recited in claim 1, wherein the liver drug comprises a hepatitis C virus-related hepatocellular carcinoma (HCV-related HCC) drug.

5. The in vitro method as recited in claim 1, wherein the liver drug comprises a drug for liver failure.

6. The in vitro method as recited in claim 1, wherein the liver drug comprises a drug for liver cancer stem cell.

7. The in vitro method as recited in claim 1, wherein the isolated human liver tumor cell line further comprises a reporter gene.

8. The in vitro method as recited in claim 7, wherein the reporter gene expresses fluorescence or luminescence.

9. The in vitro method as recited in claim 1, wherein the isolated human liver tumor cell line secretes alpha-fetoprotein and albumin.

10. The in vitro method as recited in claim 1, wherein the isolated human liver tumor cell line has a cancer stem cell potential.

11. The in vitro method as recited in claim 1, wherein the isolated human liver tumor cell line expresses CD13, CD44, and EpCAM.

12. The in vitro method as recited in claim 1, wherein the isolated human liver tumor cell line forms a spheroid-like structure of stem cell in an ultra low attachment flask.

13. The in vitro method as recited in claim 1, wherein the isolated human liver tumor cell line has carcinogenicity in severe combined immunodeficiency (SCID) mice.

14. The in vitro method as recited in claim 1, wherein the cells are placed in a medium containing 10% serum.
